(19) United States
(12) Patent Application Publication
(10) Pub. No.: US 2024/0165340 A1
(43) Pub. Date: May 23, 2024

(54) COMPOSITION INJECTION DEVICE

(71) Applicant: L&C BIO CO.,LTD., Seongnam-si Gyeonggi-do (KR)

(72) Inventors: Kee Won LEE, Seoul (KR); Hyung Gu KIM, Seoul (KR); Whan Chul LEE, Seoul (KR)

(21) Appl. No.: 18/553,107

(22) PCT Filed: Mar. 29, 2021
(86) PCT No.: PCT/KR2021/003840

§ 371 Date: Sep. 28, 2023

(30) Foreign Application Priority Data
Mar. 29, 2021 (KR) 10-2021-0040067

(51) Int. Cl. A61M 5/315

(52) U.S. Cl. CPC A61M 5/31565

(57) ABSTRACT

A composition injection device includes a needle which is provided such that the ends of a distal part and proximal part thereof are open, and which is hollow such that a composition is injected thereto or discharged therefrom; a pressing member inserted into the needle to discharge the composition from the needle by pressurizing the composition injected into the needle; and a paddle which is formed to extend from the end of the distal part of the needle, and which enables the composition discharged from the needle to be applied, wherein the maximum width of the paddle is at most the outer diameter of the needle.

### TECHNICAL FIELD

The present disclosure relates to an injection device, and more particularly, to a composition injection device which is a device for injecting a composition for tissue regeneration that allows the composition to be easily injected into a cartilage defect site and the injected composition to be evenly applied.

### BACKGROUND ART

Osteoarthritis is a chronic disease that is characterized by pain, rigidity, and failure caused by a defect in cartilage and surrounding tissue, and cases of osteoarthritis are increasing with an increase in the elderly population.

For reference, cartilage is a non-neural, avascular tissue, and once a cartilage defect occurs, it is difficult to regenerate the cartilage, and the defect occurs across a wide area. Thus, surgical treatment is required.

Examples of currently applied surgical treatment include microfracture, an osteochondral autograft transplantation system, autologous chondrocyte implantation, and the like which are methods of improving a cartilage defect lesion.

In microfracture which is the first-line treatment, washing may easily occur due to a synovial fluid or washing fluid and incomplete cartilage regeneration may occur, a blood clot may easily be detached or worn due to body weight or joint motion, and application is limited to only small cartilage defect sites. Therefore, due to the formation of fibrous cartilage instead of hyaline cartilage which is an original articular cartilage component, there is a limited effect for complete cartilage regeneration.

Meanwhile, an osteochondral autograft transplantation system or autologous chondrocyte implantation is applied to larger cartilage defect sites and has a high success rate. However, since an osteochondral autograft transplantation system or autologous chondrocyte implantation requires two different kinds of treatment, including collection of autologous cartilage or autologous chondrocytes through first-line treatment and then autografting through second-line treatment, the process of collecting autologous cartilage or autologous chondrocytes always involves the problem of a defect in normal tissue surrounding the cartilage.

Stem cell-based cartilage treatment, which has recently gained attention, is a method of injecting stem cells collected and isolated from autologous or allogeneic tissue into a cartilage defect site to induce cartilage formation through differentiation of the stem cells into chondrocytes. This treatment method can be applied to lesion sites of various areas without limitations but has limitations in that collection of autologous cells for implantation is essential and the cost of the treatment is high. Also, there are disadvantages that differentiation of the injected stem cells into normal chondrocytes is not completely guaranteed, and long-term hospitalization and rehabilitation are required after the treatment.

In order to address the above-mentioned limitations of the existing treatment methods, a treatment method in which a human-derived cartilage component is directly injected into a cartilage defect site is applied. Such a treatment method can improve cartilage regeneration by processing a human-derived cartilage component into the form of micronized powder, mixing the micronized powder with a patient's blood or platelet-rich plasma (PRP), and then injecting the mixture into a cartilage defect site.

Here, BioCartilage® (Arthrex, Inc.), which is a commercial product used for the above treatment method, is a human tissue product obtained by processing a donor's cartilage tissue into micronized powder whose average particle size is 100 to 300 μm. For reference, according to the result of a preclinical animal model study, it has been reported that when BioCartilage® is mixed with PRP and used to treat a full-thickness cartilage defect after microfracture, not only are safety and biocompatibility achieved, but also improved cartilage regeneration is exhibited compared to when microfracture is used alone.

An instrument for delivering the above-described BioCartilage® to a cartilage defect site after microfracture is disclosed in the related art, U.S. Unexamined Patent Application Publication No. 2015-0045768.

According to the related art, a delivery instrument consists of a needle with which an elevator and a paddle are combined. The needle has a distal end that is bent to be curved and has a separate paddle attached thereto, The back surface of the paddle is smoothly curved at a predetermined angle to flatten a delivered material, and a tip of the paddle serves as an elevator that is lifted to push and spread the delivered material.

However, in the delivery instrument of the related art, since the needle has a curved shape and has the paddle, which is larger than the width of the needle, attached to the distal part to protrude therefrom, there is a problem that, when the delivery instrument is inserted or removed through a portal during arthroscopic surgery, the paddle is caught around the portal. Also, since the surface of the paddle is smoothly curved, there is a disadvantage in that a composition obtained by mixing sticky, refined human-derived cartilage with blood is prone to adhere to the surface. Furthermore, since the delivery instrument does not have a connecting portion with a syringe containing the composition, there is an inconvenience in that the composition obtained by mixing the human-derived cartilage with blood should be inserted using a separate syringe or tool before being injected into a cartilage defect site.

Thus, there is a need for the development of a composition injection device that can be conveniently used by a user during arthroscopic surgery while preventing a paddle from being caught around a portal or a composition from adhering to a surface of the paddle when the composition injection device is inserted or removed through the portal during arthroscopic surgery.

### DISCLOSURE

### Technical Problem

The present disclosure is directed to providing a composition injection device that allows a composition to be injected into a cartilage defect site and the injected composition to be evenly applied on the cartilage defect site during minimal incision and arthroscopic surgery.

The present disclosure is also directed to providing a composition injection device that is easy to insert and remove through an arthroscopic portal formed in a joint during minimal incision and arthroscopic surgery.

The present disclosure is also directed to providing a composition injection device that can be directly connected to a composition-containing device in the form of a syringe or the like that contains a composition.

Objectives of the present disclosure are not limited to the above-mentioned objectives, and other unmentioned objectives should be clearly understood by those of ordinary skill in the art from the description below.

### Technical Solution

According to the present disclosure, the above objectives can be achieved by a composition injection device including a needle which is provided in a state in which an end of each of a distal part and a proximal part is open and which is provided to be hollow for a composition to be injected into the needle or discharged from the needle, a pressing member inserted into the needle to press the composition injected into the needle and cause the composition to be discharged from the needle, and a paddle formed to extend from the end of the distal part of the needle and configured to allow application of the composition discharged from the needle, wherein a maximum width of the paddle is formed to be less than or equal to an outer diameter of the needle.

According to the present disclosure, the above objectives can also be achieved by a composition injection device including a needle which is provided in a state in which an end of each of a distal part and a proximal part is open and which is provided to be hollow for a composition to be injected into the needle, a paddle provided at the end of the distal part of the needle and configured to allow application of the composition discharged from the needle, a needle handle provided at the end of the proximal part of the needle to support the needle and having a connecting portion provided thereon, and a composition-containing device coupled to the connecting portion while containing a composition therein and configured to, in response to an external force applied thereto, press the contained composition so that the contained composition is discharged through the needle, wherein a maximum width of the paddle is formed to be less than or equal to an outer diameter of the needle.

### Advantageous Effects

In a composition injection device of the present disclosure, since a maximum width of a paddle is formed to be less than or equal to an outer diameter of a needle, and the outer diameter of the needle and the width of the paddle are formed to have a smaller size than a width of an arthroscopic portal, the composition injection device can be easily inserted and removed through the arthroscopic portal to allow application of a composition on a cartilage defect site.

Also, since a protrusion is formed on a paddle in a composition injection device of the present disclosure, a composition discharged from a needle can be effectively applied on a cartilage defect site while being prevented from adhering to the paddle.

In addition, since a composition injection device of the present disclosure can be, through a connecting portion formed on a needle handle, directly connected to a composition-containing device in the form of a syringe or the like that contains a composition, usability of the composition injection device can be increased.

Advantageous effects of the present disclosure are not limited to the above-mentioned advantageous effects, and other unmentioned advantageous effects should be clearly understood by those of ordinary skill in the art to which the present disclosure pertains from the description below.

### DESCRIPTION OF DRAWINGS

FIG. 1 is an exploded perspective view of a composition injection device according to one embodiment of the present disclosure.

FIG. 2 is a lateral perspective view of the composition injection device illustrated in FIG. 1.

FIGS. 3 and 4 are enlarged views of portions of the composition injection device illustrated in FIG. 1.

FIGS. 5 and 6 are cross-sectional views for describing one example of a protrusion formed on a paddle of the composition injection device illustrated in FIG. 1.

FIGS. 7 and 8 are cross-sectional views for describing another example of the protrusion formed on the paddle of the composition injection device illustrated in FIG. 1.

FIGS. 9 and 10 are views for describing a state in which a composition is discharged through the composition injection device illustrated in FIG. 1.

FIGS. 11 and 12 are views for describing the fact that, when a composition is discharged through the composition injection device illustrated in FIGS. 9 and 10, the discharged composition can be cut using a tip of the paddle.

FIG. 13 is an exploded perspective view of a composition injection device according to another embodiment of the present disclosure.

FIG. 14 is an enlarged view of a portion of the composition injection device illustrated in FIG. 13.

FIG. 15 is a view illustrating an aspect of use of the composition injection device illustrated in FIG. 13.

### MODES OF THE INVENTION

Hereinafter, embodiments of the present disclosure will be described in detail regarding the accompanying drawings to allow those of ordinary skill in the art to which the present disclosure pertains to easily carry out the present disclosure. The present disclosure may be implemented in various forms and is not limited by the embodiments described herein.

It should be noted that the drawings are schematic and are not drawn to scale. Relative dimensions and proportions of parts in the drawings may have been exaggerated or understated for the sake of clarity and convenience, and arbitrary dimensions are only illustrative instead of limiting. Also, the same structure, element, or part shown in two or more drawings is denoted by the same reference numeral to indicate similar features.

Embodiments of the present disclosure show ideal embodiments of the present disclosure in detail. As a result, various modifications of the drawings are expected. Therefore, the embodiments are not limited to specific illustrated forms and also include modifications of forms due to manufacture, for example.

Hereinafter, composition injection devices 100 and 200 according to embodiments of the present disclosure will be described with reference to the accompanying drawings.

First, although not illustrated in the drawings, the composition injection devices 100 and 200 according to embodiments of the present disclosure are devices for applying a composition B on a cartilage defect site or removing the applied composition B through an arthroscopic portal (not illustrated) formed in a joint during minimal incision and arthroscopic surgery.

Here, the composition B injected into the composition injection devices 100 and 200 according to embodiments of the present disclosure may be a composition for tissue regeneration that is produced in the form of a sticky paste by mixing micronized acellular cartilage powder with one or more biocompatible polymers or crosslinked products of the biocompatible polymers. That is, the composition B may be a composition for cartilage regeneration.

For reference, the micronized acellular cartilage powder is cartilage powder obtained by removing human and animal-derived chondrocytes and then performing micronizing into sizes of several μm.

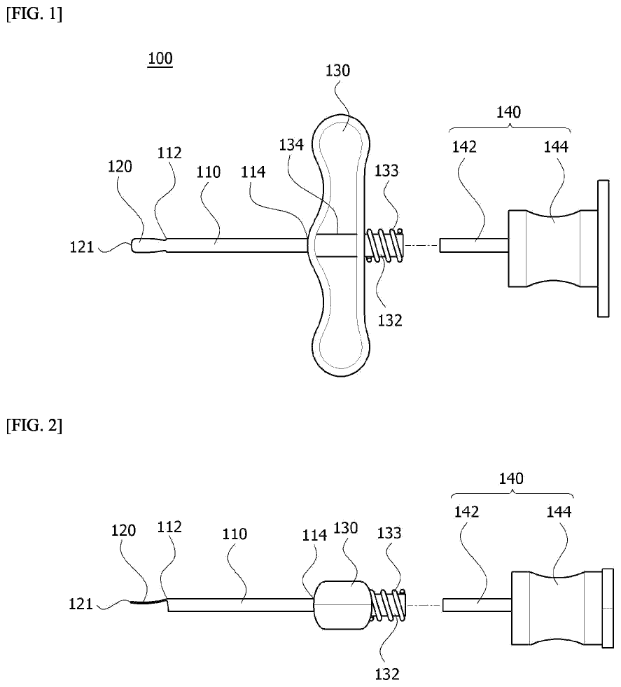

First, the composition injection device 100 according to one embodiment of the present disclosure will be described with reference to FIGS. 1 to 12.

As illustrated in FIGS. 1 and 2, the composition injection device 100 according to one embodiment of the present disclosure may include a needle 110, a pressing member 140, a paddle 120, and a needle handle 130.

Referring to FIGS. 1 and 2, the needle 110 is a part that serves as a flow path through which the composition B is injected and the injected composition B is discharged.

The needle 110 is provided in a state in which an end of each of a distal part 112 and a proximal part 115 is open.

Also, the needle 110 is provided in a linear shape to allow the injected composition B to be easily discharged.

For reference, the needle 110 according to one embodiment of the present disclosure may be made of a metal material such as stainless steel, but the present disclosure is not necessarily limited thereto.

Here, an outer diameter D1 of the needle 110 may be provided to be smaller than a width of the arthroscopic portal. In other words, the outer diameter D1 of the needle 110 may be formed to be smaller than an inner diameter of a typical arthroscopic portal formed in a joint during arthroscopic surgery.

For example, the outer diameter D1 of the needle 110 may be formed to be less than or equal to 4.5 mm in consideration of the fact that an inner diameter of a typical arthroscopic portal is 5 mm and a width of a freer elevator used for pressing the composition B is 4.5 mm.

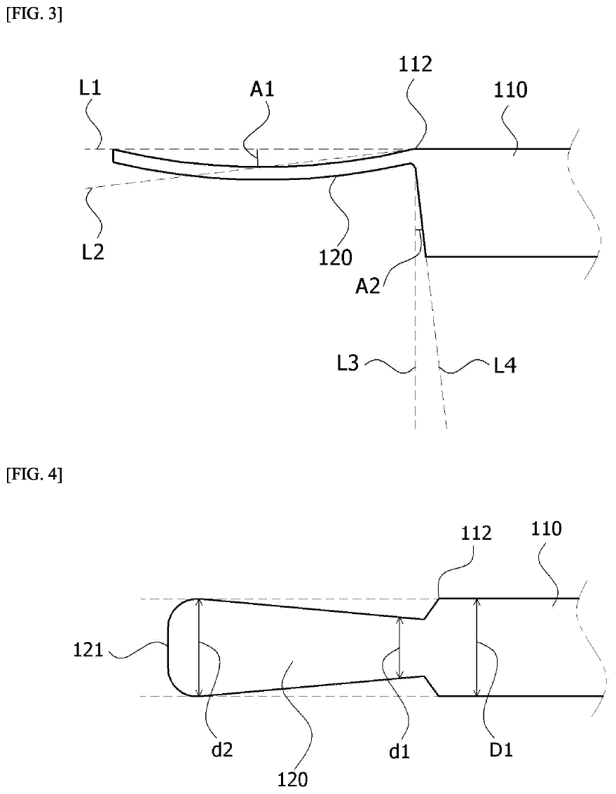

Meanwhile, referring to FIG. 3, the end of the distal part 112 of the needle 110 may be provided to be obliquely cut and have a cross-section that is inclined in one direction.

In the case in which the end of the distal part 112 of the needle 110 is obliquely cut as described above, a virtual line L4 extending along a surface obliquely conveyed from the end of the distal part 112 of the needle 10 and a virtual line L3 formed to extend in a direction perpendicular to one surface of an upper end of the needle 110 from the distal part 112 of the needle 110 may form a predetermined angle A2 with each other. The reason for obliquely cutting the end of the distal part 112 of the needle 110 as described above is to allow an inner diameter D2 of an end of a distal part 112 of the original needle 110 to have the same size as the width of the end of the distal part 112 of the needle 110 having the paddle 120 formed thereon.

Here, the predetermined angle A2 formed by the one virtual line LA and the other virtual line L3 may vary according to the slope of the cross-section of the end of the distal part 112 of the needle 110.

For example, the predetermined angle A2 may be approximately formed to be greater than 0° and less than 20°, but the present disclosure is not necessarily limited thereto.

For reference, in the case in which the predetermined angle A2 is formed to be less than 0°, due to the paddle 120 which is formed to be convex downward, the inner diameter of the end of the distal part 112 of the needle 110 is smaller than the inner diameter D2 of the original needle 110. In this case, a problem occurs in that the sticky composition B is unable to be properly discharged through the needle 110.

Conversely, in the case in which the predetermined angle A2 is formed to be greater than 20°, the inner diameter of the end of the distal part 112 of the needle 110 is larger than the diameter D2 of the original needle 110. In this case, since the sticky composition B is discharged through the needle 110 before reaching the paddle 120, a problem occurs in that an effect of pressing and spreading the composition B using the paddle 120 is reduced.

Meanwhile, referring to FIGS. 1 and 2, the needle handle 130 may be provided at the end of the proximal part 114 of the needle 110.

The needle handle 130 is for supporting the needle 110 when the composition B is discharged to the outside through the needle 110.

Here, at least one grip portion (not denoted by a reference numeral) may be formed on the needle handle 130 to facilitate gripping of the composition injection device 100 and the use thereof.

The grip portion may be formed at the needle handle 130 positioned at both sides of the needle 110 relative to the needle 110.

The grip portion may be provided to be concavely inserted in one direction to further facilitate the use of the composition injection device 100 by a user.

Meanwhile, a through-hole 134 communicating with the needle 10 is formed in the needle handle 130.

Here, a width of the through-hole 134 may be formed to be larger than or equal to the inner diameter D2 of the needle 110.

Here, the composition B injected into the needle 110 is discharged to the outside of the needle 110, that is, a cartilage defect site, by the pressing member 140.

In other words, the pressing member 140 is inserted into the needle 110 through the through-hole 134 of the needle handle 130 and allows the composition B inside the needle 110 to be discharged to the outside.

The pressing member 140 may include a push rod 142 inserted into the needle 110 through the needle handle 130 to press the composition B injected into the needle 110 and a rod handle 144 coupled to an end of the push rod 142 to press the push rod 142.

Here, the width of the push rod 142 may be formed to be smaller than the inner diameter D2 of the needle 110.

This is because the push rod 142 of the pressing member 140 should press the composition B injected into the needle 110 and cause the composition B to be discharged, and in the case in which the width of the push rod 142 is formed to be larger than the inner diameter D2 of the needle 110, the push rod 142 cannot be inserted into the needle 110.

For reference, the rod handle 144 is not limited to that illustrated in FIGS. 1 and 2 and may be formed to have the same shape as the needle handle 130.

Meanwhile, a connecting portion 132 having a fastening protrusion 133 formed on an outer peripheral surface thereof may be additionally provided on the needle handle 130.

The connecting portion 132 communicates with the through-hole 134 of the needle handle 130.

Here, the pressing member 140 may be inserted into the through-hole 134 of the needle handle 130 through the connecting portion 132.

In other words, the pressing member 140 may be directly inserted into the needle handle 130 and inserted into the needle 110 through the through-hole 134 and press the composition B inside the needle 110 so that the composition B is discharged or may be inserted into the through-hole 134 of the needle handle 130 through the connecting portion 132 and press the composition B of the needle 110 so that the composition B is discharged.

Referring to FIGS. 1 and 2, the paddle 120 is a portion for allowing the composition B discharged from the needle 110 to be applied on a cartilage defect site. The paddle 120 is formed on the end of the distal part 112 of the needle 110 and formed to extend from the distal part 112.

Widths d1 and d2 of the paddle 120 may be formed to be smaller than the inner diameter of the arthroscopic portal. Also, the widths d1 and d2 of the paddle 120 may be formed to be smaller than the outer diameter D1 of the needle 110.

For example, in the case in which the outer diameter D1 of the needle 110 is provided to be 4.5 mm or less than or equal to 4.5 mm, the widths d1 and d2 of the paddle 120 may be formed to be smaller than 4.5 mm or the outer diameter D1 of the needle 110.

Since, as described above, the outer diameter D1 of the needle 110 and the widths d1 and d2 of the paddle 120 are provided to be smaller than the width of the arthroscopic portal, and the widths d1 and d2 of the paddle 120 are provided to be less than or equal to the outer diameter D1 of the needle 110, the composition injection device 100 can be easily inserted and removed through the arthroscopic portal.

Also, as illustrated in FIG. 4, the widths d1 and d2 of the paddle 120 may be provided to, in at least one portion, gradually increase away from the distal part 112 of the needle 110.

That is, the paddle 120 may be provided in the shape of a spoon in which the width d2 at a position close to the end of the paddle 120 is formed to be larger than the width d1 at a position close to the distal part 112 of the needle 110.

Meanwhile, as illustrated in FIGS. 2 and 3, the paddle 120 for applying the composition B discharged from the needle 110 on a cartilage defect site may be formed to be bent so that at least one portion of the paddle 120 is convex in one direction.

In other words, the paddle 120 may be provided to be convex downward and flat from one surface of the upper end of the needle 110.

As illustrated in FIG. 3, at one portion of the paddle 120, a predetermined angle A1 may be formed through a virtual line L1 formed to extend from one surface of the upper end of the needle 110 and a virtual line L2 formed to pass the one portion of the paddle 120 from the distal part of the needle 110.

Here, the one portion of the paddle 120 is formed to be convex downward by as much as the predetermined angle A1 formed by the one virtual line L1 and the other virtual line L2.

Since the one portion of the paddle 120 is formed to be convexly curved downward as described above, when the composition B discharged from the needle 110 is applied on a cartilage defect site, the composition B can be more effectively pressed and spread on the cartilage defect site.

For example, the predetermined angle A1 may be approximately formed to be greater than 0° and less than 20°, but the present disclosure is not necessarily limited thereto.

Here, in the case in which the predetermined angle A1 is formed to be 0°, the paddle 120 is formed to be flat in a straight line instead of being convexly curved downward. In this case, a problem occurs in that an effect of pressing and spreading the composition B discharged from the needle 110 on the cartilage defect site using the paddle 120 is reduced.

Conversely, in the case in which the predetermined angle A1 is formed to be greater than 20°, due to the paddle 120 which is formed to be convex downward, the inner diameter of the end of the distal part 112 of the needle 110 is smaller than the inner diameter D2 of the original needle 110. In this case, a problem occurs in that the sticky composition B is unable to be properly discharged through the needle 110.

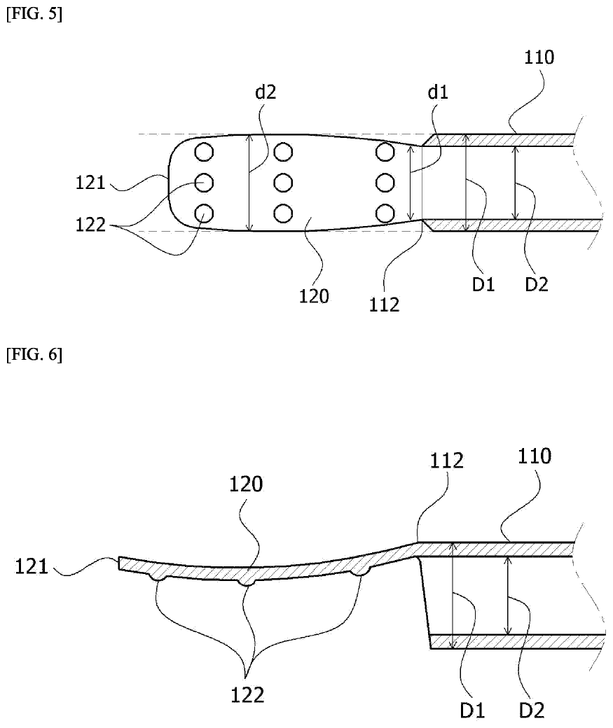

Meanwhile, referring to FIGS. 5 to 8, protrusions 122 and 122-1 may be formed on the paddle 120 according to one embodiment of the present disclosure.

One or more protrusions 122 and 122-1 may be formed to protrude from one surface of the paddle 120.

The protrusions 122 and 122-1 may be formed on a lower end surface of the paddle 120.

Here, an upper end surface of the paddle 120 may be an upper side surface connected to an upper end surface of the needle 110, and the lower end surface of the paddle 120 may be a lower side surface on which the protrusions 122 and 122-1 are formed. Also, the upper end surface of the paddle 120 may be a portion where a concave curved surface is formed, and the lower end surface of the paddle 120 may be a portion where a convex curved surface is formed.

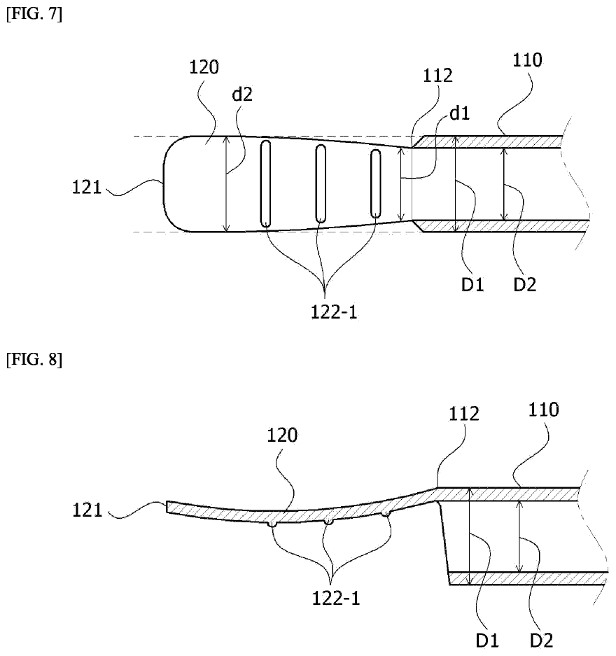

For example, as illustrated in FIGS. 5 and 6, the protrusions 122 formed on the paddle 120 may be formed as a dot-type. Also, as illustrated in FIGS. 7 and 8, the protrusions 122-1 formed on the paddle 120 may be formed as a line-type.

For reference, the forms of the protrusions 122 and 122-1 formed on the paddle 120 are not limited thereto, and the protrusions 122 and 122-1 may be formed in other forms.

The protrusions 122 and 122-1 formed as a dot-type or line-type may be provided as a plurality of protrusions 122 and 122-1.

The plurality of protrusions 122 and 122-1 may be formed to be spaced apart at predetermined intervals on one surface of the paddle 120.

Here, the plurality of protrusions 122 and 122-1 may be provided at equal intervals or provided at different intervals, but the present disclosure is not necessarily limited thereto.

Also, the plurality of protrusions 122 and 122-1 may be arranged in the horizontal direction or arranged in the vertical direction relative to one surface of the paddle 120.

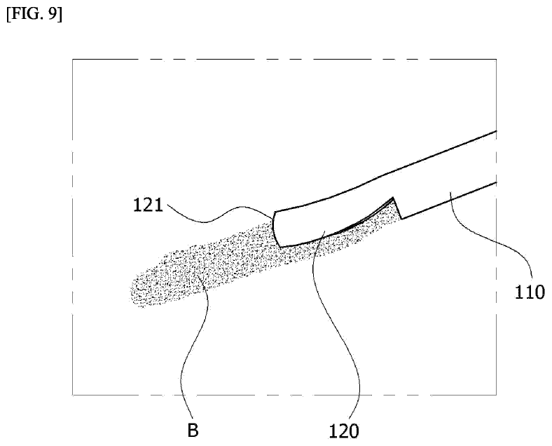

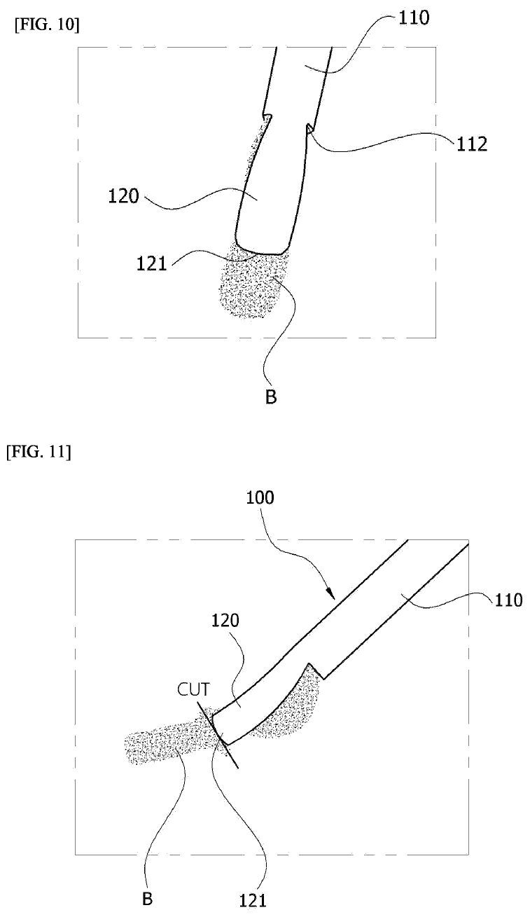

As illustrated in FIG. 9, since the protrusions 122 and 122-1 are formed on the paddle 120, the sticky composition B can be prevented from adhering to the paddle 120 while being continuously discharged through the needle 110. Further, as illustrated in FIG. 10, the composition B can be more effectively pressed and spread on a cartilage defect site.

Meanwhile, a tip 121 may be formed at the end of the paddle 120.

The tip 121 of the paddle 120 may be formed to have a sharp edge.

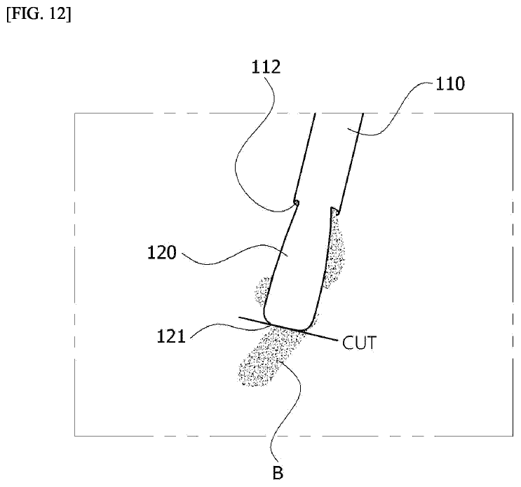

As illustrated in FIGS. 11 and 12, due to the tip 121 of the paddle 120 having a sharp edge, cutting of the composition B discharged through the needle 110 after a necessary amount of the composition B is discharged to a cartilage defect site can be easily performed.

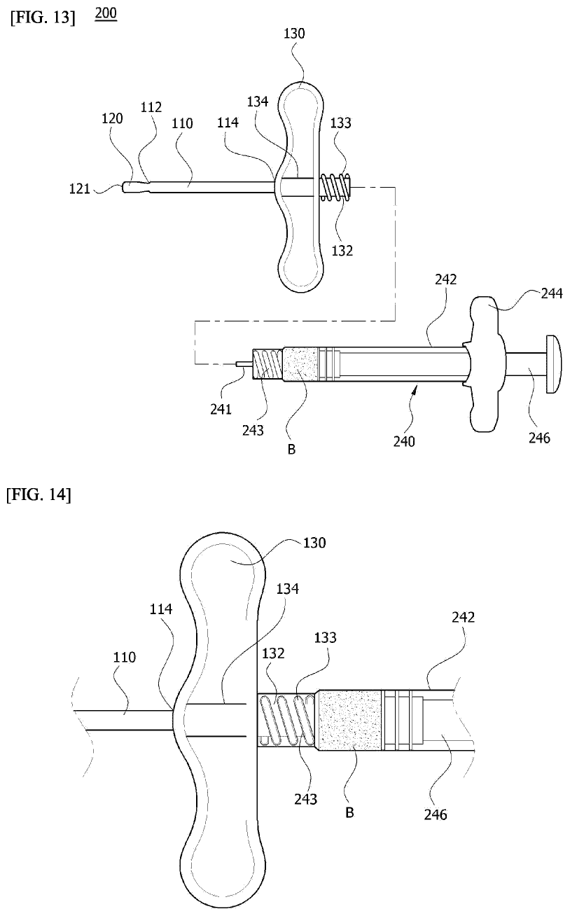

Hereinafter, the composition injection device 200 according to another embodiment of the present disclosure will be described with reference to FIGS. 13 to 15 by mainly describing differences from the above-described embodiment.

The composition injection device 200 according to another embodiment of the present disclosure is substantially the same as the composition injection device 100 according to the above-described embodiment except for including a composition-containing device 240 instead of the pressing member 140, and thus the same names and reference numerals are imparted to the same components, and descriptions of the above-described embodiment applies thereto.

Referring to FIGS. 13 and 14, the composition injection device 200 according to another embodiment of the present disclosure may include a needle 110, a paddle 120, a needle handle 130, and the composition-containing device 240.

The paddle 120 may be provided at an end of a distal part 112 of the needle 110, and the needle handle 130 may be provided at an end of a proximal part 114 of the needle 110.

Here, a connecting portion 132 having a fastening protrusion 133 formed on an outer peripheral surface thereof may be additionally provided on the needle handle 130.

The composition-containing device 240 may be coupled to the connecting portion 132 according to another embodiment of the present disclosure.

While containing a composition B therein, the composition-containing device 240 is coupled to the fastening protrusion 133 of the connecting portion 132 by a fastening groove 243 formed on an inner peripheral surface thereof.

As a result, the composition-containing device 240 is coupled to the needle handle 130 by the connecting portion 132.

As illustrated in FIGS. 13 to 15, the composition-containing device 240 in the form of a syringe may be directly connected and coupled to the connecting portion 132 of the composition injection device 200 according to another embodiment of the present disclosure.

For reference, unlike in the drawings, instead of the composition-containing device 240 in the form of a syringe, another tool or device that can contain the composition B therein and press the contained composition B may be coupled to the connecting portion 132, and the present disclosure is not necessarily limited to the above.

Meanwhile, in the composition injection device 200 according to another embodiment of the present disclosure, the composition-containing device 240 in the form of a syringe may include a delivering portion 241, a device body 242, a device handle 244, and a plunger 246.

The device body 242 is a portion for containing the composition B therein.

To this end, the device body 242 has a cylindrical shape, and an empty space is provided therein.

The delivering portion 241 is provided at one end of the device body 242.

The delivering portion 241 is a type of passage and is a portion for delivering the composition B contained in the device body 242 to the needle 110 by an external force applied to the composition-containing device 240.

Here, the fastening groove 243 is formed on an inner peripheral surface of one end of the device body 242, and the device handle 244 is provided at the other end of the device body 242. Also, the plunger 246 for pressing the composition B contained in the device body 242 and causing the composition B to move to the needle 110 is inserted into and coupled to the other end of the device body 242.

Here, when the fastening groove 243 of the device body 242 and the fastening protrusion 133 of the connecting portion 132 are coupled, the delivering portion 241 communicates with a through-hole 134 of the needle handle 130 through the inside of the connecting portion 132.

Accordingly, in the case in which the composition B contained in the device body 242 is pressed using the plunger 246 by an external force, the pressed composition B is moved to the inside of the needle 110 through the delivering portion 241, and as a result, discharged to the outside of the needle 110.

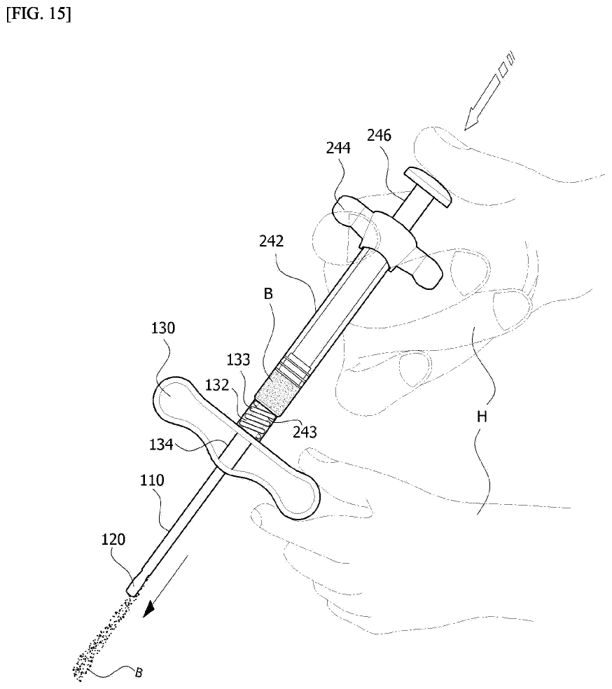

An aspect of use of the composition injection device 200 according to another embodiment of the present disclosure will be briefly described with reference to FIG. 15.

A user uses the composition injection device 200 according to another embodiment of the present disclosure with both hands.

First, the user couples the composition-containing device 240 in the form of a syringe, which contains the composition B therein, to the connecting portion 132 of the needle handle 130.

For example, the user holds the needle handle 130 and supports the needle 110 with one hand H and holds the device handle 244 of the composition-containing device 240 and presses the plunger 246 with the other hand H.

Then, composition B contained in the composition-containing device 240 is injected into the needle 110 through the delivering portion 241 by the plunger 246. Here, the composition B injected into the needle 110 is discharged to the outside of the needle 110 and applied to a cartilage defect site.

Here, the composition B applied on the cartilage defect site is spread on the cartilage defect site using the paddle 120.

By the above-described configurations, in the composition injection devices 100 and 200 according to the embodiments of the present disclosure, the outer diameter D1 of the needle 110 and the widths d1 and d2 of the paddle 120 are formed to be smaller than the width of the arthroscopic portal, and thus the composition injection devices 100 and 200 can be easily inserted and removed through the arthroscopic portal, and by the protrusions 122 and 122-1 formed on the paddle 120, the composition B discharged from the needle 110 can be effectively applied to a cartilage defect site while being prevented from adhering to the paddle 120.

Also, in the composition injection device 200 according to an embodiment of the present disclosure, since the composition-containing device 240 in the form of a syringe that contains the composition B can be directly connected to the composition injection device 200 through the connecting portion 132 formed on the needle handle 130, usability of the composition injection device 200 can be increased.

Embodiments of the present disclosure have been described above using specific details, such as specific components, and the accompanying drawings, but the embodiments are only provided for better understanding of the present disclosure, and the present disclosure is not limited to the above-described embodiments. Those of ordinary skill in the art to which the present disclosure pertains may make various modifications and changes from the above description. Therefore, the spirit of the present disclosure should not be defined by being limited to the embodiments described herein, and not only the claims, but also their equivalents and anything with equivalent modifications fall within the scope of the spirit of the present disclosure.

### INDUSTRIAL APPLICABILITY

According to an injection device relating to one embodiment of the present disclosure which is a composition injection device, a composition can be easily injected into a cartilage defect site in particular, and the injected composition can be evenly applied.

1. A composition injection device comprising:
a needle which is provided in a state in which an end of each of a distal part and a proximal part is open and which is provided to be hollow for a composition to be injected into the needle or discharged from the needle;
a pressing member inserted into the needle to press the composition injected into the needle and cause the composition to be discharged from the needle; and
a paddle formed to extend from the end of the distal part of the needle and configured to allow application of the composition discharged from the needle,
wherein a maximum width of the paddle is formed to be less than or equal to an outer diameter of the needle

2. The composition injection device of claim 1, wherein, in at least one portion, a width of the paddle is formed to gradually increase away from the distal part of the needle.

3. The composition injection device of claim 1, wherein one portion of the paddle is bent to have a shape that is convex in one direction.

4. The composition injection device of claim 3, wherein the one portion of the paddle having the convex shape is provided to form a predetermined angle with a virtual line formed to extend from one surface of an upper end of the needle.

5. The composition injection device of claim 3, wherein one or more protrusions are formed to protrude from one surface of the paddle.

6. The composition injection device of claim 5, wherein the protrusions are formed as at least one of a dot-type and a line-type.

7. The composition injection device of claim 5, wherein:
the protrusions are provided as a plurality of protrusions; and
the plurality of protrusions are formed to be spaced apart at predetermined intervals on one surface of the paddle.

8. The composition injection device of claim 1, wherein a tip that enables cutting of the composition discharged through the needle is formed at an end of the paddle.

9. The composition injection device of claim 1, wherein the end of the distal part of the needle is provided to be obliquely cut and have a cross-section that is inclined in one direction.

10. The composition injection device of claim 1, further comprising a needle handle provided at the end of the proximal part of the needle and configured to support the needle into which the pressing member is inserted,
wherein a grip portion is formed on an outer portion of the needle handle.

11. The composition injection device of claim 10, wherein:
a through-hole communicating with the needle is formed in the needle handle; and
a width of the through-hole is formed to be larger than or equal to an inner diameter of the needle.

12. The composition injection device of claim 11, wherein:
a connecting portion is additionally provided on the needle handle;
an inside of the connecting portion communicates with the through-hole of the needle handle; and
a composition-containing device having a composition contained therein and configured to cause the contained composition to be discharged through the needle in response to an external force applied thereto is connected to the connecting portion.

13. The composition injection device of claim 11, wherein the pressing member includes:
a push rod inserted into the needle through the needle handle to press the composition injected into the needle; and
a rod handle coupled to an end of the push rod to press the push rod.

14. The composition injection device of claim 13, wherein a width of the push rod is formed to be smaller than the inner diameter of the needle.

15. A composition injection device comprising:
a needle which is provided in a state in which an end of each of a distal part and a proximal part is open and which is provided to be hollow for a composition to be injected into the needle;
a paddle provided at the end of the distal part of the needle and configured to allow application of the composition discharged from the needle;
a needle handle provided at the end of the proximal part of the needle to support the needle and having a connecting portion provided thereon; and
a composition-containing device coupled to the connecting portion while containing a composition therein and configured to, in response to an external force applied thereto, press the contained composition so that the contained composition is discharged through the needle,
wherein a maximum width of the paddle is formed to be less than or equal to an outer diameter of the needle.
